(19) United States
(12) Patent Application Publication
(10) Pub. No.: US 2013/0115601 A1
(43) Pub. Date: May 9, 2013

(54) TISSUE TYPING ASSAYS AND KITS

(71) Applicants: Michael Bunce, Bromborough (GB); Benjamin John Passey, Bromborough (GB); David Charvill, Bromborough (GB)

(72) Inventors: Michael Bunce, Bromborough (GB); Benjamin John Passey, Bromborough (GB); David Charvill, Bromborough (GB)

(21) Appl. No.: 13/697,814

(22) PCT Filed: May 16, 2011
(86) PCT No.: PCT/GB2011/050930

§ 371 Date: Jan. 24, 2013

(30) Foreign Application Priority Data
May 14, 2010 (GB) 1008125.5

(51) Int. Cl. C12Q 1/68

(52) U.S. Cl. 435/6.11

(57) ABSTRACT

The present invention relates generally to compositions of lyophilised reagents suitable for nucleic acid amplification use in in-vitro diagnostics. More particularly, the invention relates to lyophilised PCR reagent compositions and methods for genotyping including HLA and/or ABO and/or HFE typing.

### CROSS REFERENCE TO RELATED APPLICATIONS

The present application is a National Stage Application claiming the priority of co-pending PCT Application No. PCT/GB2011/050930 filed May 16, 2011, which in turn claims priority from Great Britain Application No. 1008125.5 filed May 14, 2010. Applicants claim the benefits of 35 U.S.C. Section 120 as to the PCT application and priority under 35 U.S.C. Section 119 as to the Great Britain application, and the entire disclosures of both applications are incorporated herein by reference in their entireties.

### FIELD OF THE INVENTION

The present invention relates generally to compositions of lyophilised reagents suitable for nucleic acid amplification use in in-vitro diagnostics. More particularly the invention relates to lyophilised PCR reagent compositions and methods for genotyping including HLA and/or ABO and/or HFE typing.

### BACKGROUND TO THE INVENTION

Standard molecular tests used for diagnostics or life science research use a mix of components used for target amplifications in assays using techniques such as PCR, qPCR, NASBA, SDA, TMA. The assays utilise enzymes that are inherently unstable and thus are traditionally stored and transported at −20 C. The other reagents needed for the assays usually require refrigeration. Thus a kit for carrying out any molecular assay such as a test for pathogens, viruses, or genotyping assays will include multiple vials of different reagents that have different storage requirements.

This is inconvenient for larger laboratories and particularly difficult for smaller ones that have limited storage and also results in assays with many more protocol steps as they have to combine the various different reagents separately and an increased possibility of carry over. Independent of the DNA and RNA targets the reagents used in molecular amplification techniques and broadly similar and genotyping is used as one example.

Several publications and patent documents are referenced in this application in order to describe more fully the state of the art to which this invention pertains. The disclosure of each of these publications is incorporated herein by reference.

The ultimate goal of matching a donor organ is identification of an organ that will be tolerated indefinitely by the body of the recipient. Donor and recipient matching may be divided into several aspects including blood type matching and tissue type matching.

The first tier of tissue typing is blood type matching. Blood group matching is important both in relation to blood transfusions and tissue transplantation.

Human blood may be categorised into four major blood groups—A, B, AB and O. The respective blood group is determined by the type of glycoproteins present on the surface of a persons blood cells. Glycoproteins are a combination of sugar and protein. Type A cells carry type A glycoproteins whilst type B cells carry type B glycoproteins. Type AB cells have a mixture of both A and B glycoproteins. Type O cells have neither. Each person naturally has antibodies to the glycoproteins their own cells lack and it is these antibodies that are responsible for causing serious reactions to incompatible blood.

People with type A cells have antibodies to type B glycoproteins. Thus, blood or tissue from a type B donor will not be compatible with a type A recipient. Similarly, people having type B cells have antibodies to type A glycoproteins, and will not be compatible with a type A donor. AB individuals lack antibodies to type A and type B glycoproteins and are therefore potentially compatible with any donor. In contrast, individuals that are type O have antibodies against both type A and type B glycoproteins and therefore require type O donors. With regard to blood group, people with blood type AB are called universal recipients whilst those with blood type O are known as universal donors.

The second tier of tissue typing is tissue matching. This involves testing the similarity of various proteins/antigens between a donor and a potential recipient. Similarity may also be determined through the use of blood tests.

Tissue matching involves looking at major histocompatibility complex or HLA antigens. By analysing which of these specific antigens both individuals have, it is possible to determine the closeness of tissue matching.

The human leukocyte antigen system (HLA) is the name of the major histocompatibility complex (MHC) in humans. The function of major histocompatibility complex (MHC) molecules is to ‘present’ antigenic peptides to T-cells of the immune system. The peptides, which are around nine amino acids in size are bound within a groove of the MHC molecule by non-covalent forces.

The HLA locus contains a large number of genes related to immune system function. This group of genes reside on chromosome 6, and encode cell-surface antigen-presenting proteins and many other genes. The HLA genes are the human versions of the MHC genes that are found in most vertebrates. The proteins encoded by certain genes are also known as antigens, as a result of their historic discovery as factors in organ transplantations. The major HLA antigens are essential elements for immune function. Different classes have different functions:

HLA Class I molecules comprise a transmembrane protein attached to a molecule of β-2-microglobulin and a short peptide. MHC Class I molecules are expressed at the surface of almost all cells in the body. Class I antigens (A, B & C) present peptides from inside the cell (including viral peptides if present). These peptides are produced from digested proteins that are broken down in the proteasomes. The peptides are generally small polymers, about 9 amino acids in length. Foreign antigens attract killer T-cells (also called CD8 positive- or cytotoxic T-cells) that destroy cells. Classical HLA Class I antigens are encoded by the genes HLA-A, HLA-B and HLA-C.

HLA class II molecules comprises two transmembrane polypeptides—an α chain and a β chain. Generally these molecules are expressed where inflammation occurs. The classical Class II molecules present antigens (DP, DM, DOA, DOB, DQ, & DR) from outside of the cell to T-lymphocytes. These particular antigens stimulate T-helper cells to multiply, and these T-helper cells then stimulate antibody-producing B-cells to produce Antibodies to that specific antigen. Self-antigens are suppressed by suppressor T-cells. Class II antigens are encoded by HLA-D genes.

The HLA genes are polymorphic—as of November 2009 there were: 309 ‘A’ Alleles, 563 ‘B’ Alleles, 167 ‘C’ Alleles. With regard to HLA-D, there were: 20 α chain and 107 β chain ‘DP’ Alleles, 3 α chain and 439 β chain ‘DR’ Alleles and 25 α chain and 56 β chain ‘DQ’ Alleles.

Currently tissue matching involves looking at six major histocompatibility complex or HLA antigens. A six-antigen match is the best compatibility between a donor recipient pair who are not identical twins. This occurs 25% of the time between siblings having the same mother and father and also occurs from time-to-time in the general population. FIGS. 1 and 2 demonstrate the chromosomal regions relevant to HLA and ABO genotyping.

Long-term outcomes in transplantation generally do not relate to matching. However, analysis of thousands of transplants consistently shows that six-antigen matched organs have the best statistical results, followed progressively by five antigens, and then four antigens, etc. For this reason, when a close match is available, it is preferred. Thus, whilst other factors such as the patient's age also affect the results of transplantation, being able to more accurately and quickly tissue type organs may contribute to a successful therapeutic outcome.

In order to improve efficiency, Allele specific Polymerase Chain Reaction techniques such as Allele Specific Amplification (ASA), Amplification Refractory Mutation System (ARMS) and Single Specific Prime-PCR (SSP) have been applied to ABO and/or HLA genotyping. The respective methods have been popularised as being rapid and relatively easy methods of genotyping. Whilst such techniques allow the detection of cis-located polymorphisms, i.e. those located on the same chromosome, some are currently only useful when applied to polymorphisms that are located at least 80 nucleotides apart, up to a maximum of 1000 bp apart. However, identifying Single Nucleotide Polymorphisms (SNPs) that are closer than 80 bp apart is difficult because the PCR amplicons are too small to be readily identified by standard gel electrophoresis.

In addition, not all PCR templates amplify with equal efficacy. For example, it is well known that templates having a high GC content, or poly GC regions are difficult to amplify. Amplification of HLA alleles is difficult because they comprise introns having GC rich sequences that can cause amplification failure and false negative reactions to occur.

Further, the highest resolution HLA typing is currently obtained with fluorescent, Sanger-based DNA sequencing using capillary electrophoresis. However, ambiguities in HLA typing data persist due to multiple polymorphisms between alleles and resultant phase ambiguities when both alleles are amplified and sequenced together. Haplotypes are present in any given diploid organism. This is due to the frequent occurrence of multiple heterozygous sites in a given individual. With two heterozygous sites there are four possible haplotypes and the number of possible haplotypes doubles for each additional heterozygous site in the individual. Ideally, an individual's genotype across multiple heterozygous loci should include resolution into the two constituent haplotypes. Resolving these ambiguities requires time-consuming approaches such as multiple PCR amplification reactions and then analyzing the two alleles separately.

Numerous methods have been described to enable effective discrimination of genotypes, for example PCR restriction assays, real-time PCR with fluorescent probes, SYBR Green® melting curve and the like. Such methods are often overly complex, not always accurate and require multiple steps and optimisation to put into practice. In addition, whilst methods such as real-time PCR and melting curve analysis detect the amount of final amplified product, they do not detect or visualise the size of an amplified product directly, such information being obtained indirectly through use of fluorescence measurement or melting temperature data.

Thus, there is a need for genotyping compositions that are storage stable and permit rapid and efficient genotyping results to be obtained.

The present inventors have found that it is possible to provide lyophilised compositions containing all the components necessary to conduct an amplification reaction, such as a PCR assay used for genotyping. A lyophilised reaction mixture simplifies the process of preparing amplification reaction mixtures—all the necessary components, with the exception of template nucleic acid and water, can be provided in a single vessel. Lyophilisation also means that the compositions may be kept at room temperature rather than under special storage conditions such as in a fridge or freezer.

The present inventors have also been able to prepare lyophilised compositions containing all the reagents necessary to carry out Real Time Polymerase Chain Reaction (RT-PCR) assays.

Real Time Polymerase Chain Reaction (RT-PCR) is a modified PCR technique which measures the relative quantity of PCR product formed during each cycle of the PCR process. The basic principle and materials used in RT-PCR are the same as standard PCR, with the addition of reporting chemistries, typically fluorescent ones, that allow the detection of amplified product the reaction.

The reporting chemistries are either non-specific, for example an intercalating dye such as SYBR® green, whose fluorescence increases with an increase in the amount of amplified product in each round of PCR, but is indiscriminate to which DNA it binds to; or specific, utilising probe based chemistry such as TaqMan® probes, Scorpion® probes or Molecular Beacons, which are designed only to bind to defined PCR products, or non-probe based chemistries such as Plexor®.

RT-PCR offers numerous advantages over standard PCR—it is a closed-tube system, with no post-PCR manual steps such as gel electrophoresis. It measures the relative quantity of PCR product, and the efficiency of PCR. As it is much more sensitive than standard end-point detection PCR, smaller quantities of template are required.

Real time PCR is a closed tube system, therefore, the use of a lyophilised complete reaction in the assay is especially beneficial as all the user has to do is add DNA and perform the real time PCR reaction, therefore, creating a stable one-step assay.

### SUMMARY OF THE INVENTION

In a first aspect the invention provides a composition for use in a reaction for the amplification and/or synthesis of at least one polynucleotide comprising
(i) a polymerase enzyme;
(ii) dNTPs;
(iii) a cryoprotectant;
(iv) a stabilising salt;
(v) at least one primer; and
(vi) optionally a buffering agent.

The polymerase enzyme may be an RNA or DNA polymerase, particularly a DNA polymerase selected from Klenow fragment, T4 DNA polymerase, heat-stable DNA polymerases from a variety of thermostable bacteria (such as Taq, VENT, Pfu, TfI DNA polymerases) as well as their genetically modified derivatives (TaqGold, VENTexo, Pfu exo) The stabilising salt may be ammonium sulphate, potassium chloride, magnesium chloride, or the like.

Ammonium sulphate is particularly useful in compositions used for genotyping assays.

The compositions of the present invention may be genotyping compositions for use in a reaction for the amplification and/or synthesis of at least one polynucleotide. In particular embodiments the genotyping composition is for HLA and/or ABO and/or HFE genotyping, more particularly the genotyping composition is for use in a PCR reaction.

Particular compositions of the present invention comprise
(i) a Taq DNA polymerase enzyme;
(ii) at least one nucleotide selected from the group consisting of dATP, dCTP, dTTP, dGTP and dUTP;
(iii) a cryoprotectant;
(iv) ammonium sulphate
(v) at least one genotyping primer; and
(vi) optionally a buffering agent.

Preferably the composition(s) of the present invention is/are a lyophilised composition.

In certain embodiments the ammonium sulphate is at a concentration of between 0.05 M and 2M.

In certain embodiments the composition(s) comprise a buffering agent selected from the group consisting of Tris-HCl and Tween.

Particularly the composition(s) of the present invention comprise a cryoprotectant. More particularly the cryoprotectant is Trehalose di-hydrate.

The composition(s) of the invention comprise at least one primer, which may, for example, be a genotyping primer or a diagnostic primer. More particularly the at least one primer (including a genotyping or diagnostic primer) comprises a template binding region and a deliberately target-mismatched tail region. In certain embodiments the tail region is a nucleotide sequence of between 5-100 nucleotides in length. In other embodiments the tail region is a moiety that increases the molecular weight of the primer. In yet other embodiments the tail region is a positively charged moiety. In still yet other embodiments the tail region is a negatively charged moiety.

In particular embodiments the moiety that increases the molecular weight of the primer comprises an amine group. In other embodiments the moiety that increases the molecular weight of the primer is a carbon chain, more particularly a carbon chain that is between 12 and 18 units long.

Preferably the genotyping composition is a multiplex genotyping composition. More preferably the genotyping composition is a multiplex genotyping composition that comprises at least four primers generating at least 2 differently sized amplicons, more particularly such primers are for amplifying any polymorphic gene with greater than 2 polymorphisms such as, by way of non-limiting example, an HLA gene and/or ABO gene. In particular embodiments the HLA gene(s) is/are HLA-A, HLA-B, HLA-C, HLA-DRB1, DRB3, DRB4, DRB5, DQB1, DQA1, DPA1, DPB1 or any of the polymorphic genes in the MHC cluster on chromosome 6.

In particular embodiments the genotyping primers are selected from the sequences shown in the following text, tables, figures and examples.

In certain embodiments the genotyping composition comprises:
(i) 0.5 units of a Taq DNA polymerase enzyme;
(ii) 0.25 mM dNTPs comprising dATP, dCTP, dTTP. dGTP;
(iii) 5% w/v Trehalose di hydrate as a cryprotectant;
(iv) 0.2M ammonium sulphate
(v) at least one genotyping primer; and
(vi) the buffering agents Tris-HCl and Tween.

In particular embodiments the at least one primer comprises a template binding region and a tail region.

In certain embodiments the genotyping composition comprises:
(i) a Taq DNA polymerase enzyme;
(ii) at least one nucleotide selected from the group consisting of dATP, dCTP, dTTP. dGTP and dUTP;
(iii) a cryoprotectant;
(iv) at least one genotyping primer; and
(v) optionally a buffering agent,
wherein the at least one primer comprises a template binding region and a tail region.

In aspects comprising the modified genotyping primer(s) the lyophilised composition may be prepared with a buffer that does not comprise ammonium sulphate. In particular embodiments of the invention the lyophilised compositions comprise both ammonium sulphate and at least one modified genotyping or diagnostic primer(s).

The compositions of the present invention may be used in to perform standard PCR amplification, hot-start PCR, or a real-time PCR amplification, also called quantitative real time polymerase chain reaction, Q-PCR, qPCR, qrt-PCR or kinetic polymerase chain reaction (KPCR).

Thus the compositions of the present invention may include additional reagents needed to carry out a real-time PCR, for example, a reporter reagent in the form of a non-specific intercalating dye, a specific probe, or a specific, non-probe based reporter reagent such as used in the Plexor® assay.

The lyophilised compositions of the present invention may be stable at room temperature for at least 12 months. The compositions may also be stable at elevated temperature, for example 37° C. for at least 12 months.

In a second aspect of the invention there is provided a method for detecting the presence or absence of at least one variant nucleotide in one or more nucleic acids contained in an aqueous sample, comprising, treating the aqueous sample with appropriate nucleoside triphosphates, a Taq DNA polymerase and at least one diagnostic primer for a diagnostic portion of target base sequence under hybridising conditions, the nucleotide sequence of the at least one diagnostic primer being such that it is substantially complementary to the diagnostic portion, the 5′ or 3′ terminal nucleotide of the diagnostic primer being either complementary to the suspected variant nucleotide or to the corresponding normal nucleotide, whereby an extension product of the diagnostic primer is synthesised when the said terminal nucleotide of the diagnostic primer is complementary to the corresponding nucleotide in the target base sequence, no extension product being synthesised when the said terminal nucleotide of the diagnostic primer is not complementary to the corresponding nucleotide in the target base sequence, and detecting the presence or absence of the suspected variant nucleotide from the presence or absence of an extension product, characterised in that the nucleoside triphosphates, Taq DNA polymerase and diagnostic primer are in lyophilised form and the diagnostic primer comprises a tail region that increases the size of an extension product synthesised but which is non-complementary to the nucleotide sequence.

In particular embodiments of the second aspect the method comprises, (i) treating the sample with appropriate nucleoside triphosphates, a Taq DNA polymerase, a diagnostic primer for a diagnostic portion of a target base sequence and a corresponding amplification primer under hybridising conditions, the nucleotide sequence of the said diagnostic primer being such that it is substantially complementary to the said diagnostic portion, the 5′ or 3′ terminal nucleotide of the diagnostic primer being either complementary to the suspected variant nucleotide or to the corresponding normal nucleotide, whereby an extension product of the diagnostic primer is synthesised when the said terminal nucleotide of the diagnostic primer is complementary to the corresponding nucleotide in the target base sequence, no extension product being synthesised when the said terminal nucleotide of the diagnostic primer is not complementary to the corresponding nucleotide in the target base sequence, any extension product of the diagnostic primer formed being capable of serving as a template for synthesis of an extension product of the said amplification primer after separation from its complement, (ii) treating the sample under denaturing conditions to separate the primer extension product from as template where such extension product is formed, (iii) contacting single strands produced in step (ii) with appropriate nucleoside triphosphates, a Taq DNA polymerase, a diagnostic primer and an amplification primer as herein defined whereby, where possible, to synthesise further extension products using the single strands produced In step (ii) as templates (iv) repeating steps (ii) and (iii) a sufficient number of times to result in detectable amplification of the appropriate nucleotide sequence, and (v) detecting the presence or absence of the suspected variant nucleotide from the presence or absence of an amplification product obtained in step (iv).

In particular embodiments of the second aspect the tail region is at the 5′ end of the diagnostic primer and wherein the terminal nucleotide of the diagnostic primer being either complementary to the suspected variant nucleotide or to the corresponding normal nucleotide is at the 3′ end of the diagnostic primer.

In a third aspect of the invention there is provided HLA and/or ABO genotyping kits comprising lyophilised compositions according to the first aspect of the invention. Preferably such kits are for use according to the second aspect of the invention.

In particular embodiments the kits include at least one nucleotide that comprises a thermally labile 3′-substitution group.

In a fourth aspect of the invention there is provided the use of ammonium sulphate in the preparation of a lyophilised PCR composition, particularly a lyophilised PCR genotyping composition. There is also provided the use of at least one primer which comprises a template binding region and a tail region to increase the size of at least one amplicon in a PCR genotyping reaction. In particular embodiments the at least one primer is provided in a lyophilised composition and yet more particularly the lyophilised composition further comprises ammonium sulphate.

### BRIEF DESCRIPTION OF THE DRAWINGS

FIG. 1 illustrates Polymorphic STR markers located throughout the HLA region, on chromosome 6. The STRs are ordered from telomere (top) to centromere (bottom) and their position is compared with genes of the HLA complex. D6S299, D6S276 and D6S426 markers are located outside the HLA region. All STR markers are di-nucleotide repeats, except for RF that is tri-nucleotide repeats, and MOG-TAAA, D6S2414, D6S2415 and D6S497 that are tetra-nucleotide repeats. Mb=Mega bases. The HLA region spans 4×10⁶ nucleotides on chromosome 6p21.1 to p21.3, with class II, class III and class I genes located from the centromeric (Cen) to the telomeric (Tel) end. HLA class I molecules restrict CD8+ cytotoxic T lymphocyte function and mediate immune responses against ‘endogenous’ antigens and virally infected targets, whereas HLA class II molecules are involved in the presentation of ‘exogenous’ antigens to T helper cells. The HLA class III region contains many genes encoding proteins that are unrelated to cell-mediated immunity but that nevertheless modulate or regulate immune responses in some way, including tumour necrosis factor (TNF), heat shock proteins (Hsps) and complement proteins (C2, C4). The full genetic sequence of these regions is available, for example from NCBI at http://www.ncbi.nlm.nih.gov/sites/genome.

FIG. 2 illustrates a portion of human chromosome 9. 9q34 is the location, or locus of the ABO gene, which codes for the ABO blood group. It is also the location of many other genes that may be associated with the ABO blood group of an individual through genetic linkage. The full genetic sequence of this region is available, for example from NCBI at http://www.ncbi.nlm.nih.gov/sites/genome.

FIG. 3 demonstrates the results of PCR amplification using either ammonium sulphate buffer or a potassium chloride based buffer. The results demonstrate that only the ammonium sulphate buffer was competent in amplifying the 547 bp cross intron amplification. Whilst the KCl buffer was sufficient for amplifying the 1000 bp control and a short amplicon indicating the presence of B*58, lane 3 demonstrates that the KCl buffer did not amplify the B*37 fragment, a cross intron amplification.

FIG. 4a shows a virtual image of the products of a PCR reaction when amplicons were size separated on a Shimadzu MultiNA system.

FIG. 4b is a graph showing the size of the peaks of the virtual image as given by the MultiNA.

FIG. 5 illustrates the design, use and implementation of artificially extended primers to generate PCR amplicons that are visually improved to generate suitable genotyping primer combinations.

FIG. 6: Extended primers and the multiplexing method were applied to the detection of the alleles associated with Coeliac disease. Coeliac disease is associated with the HLA allele groups DQA1*05, DQB1*02 and DQB1*0302. Presence of these in cis or trans orientation can result in autoimmune response to gluten and consequently Coeliac disease. The aim of this variably extended PCR Multiplex experiment was to design a single PCR that identifies DQB1*02 and *0302 with DQA*05 capillary gel electrophoresis for the detection of the by fragments.

FIG. 7: Fifteen DNA samples from well classified reference cell lines were amplified using the standard protocols for freeze-dried SSP mixes. The primers used in each mix were from the multiplexed Coeliac kit, namely primer IDs A550, A551, A552, A556 and A558. Each mix also contained a pair of primers to amplify a housekeeping gene as a positive control for the PCR (expected size 710 bp). The fifteen amplicons were subject to electrophoresis on the Lab901 screentape DX8 system along with a ladder (bands at 25, 100, 200, 400, 600, 800

FIG. 8 shows the results obtained with lyophilised RT-PCR and “wet” RT-PCR in an assay designed to specifically identify HLA-B*27.

FIG. 9 shows the results of stability testing of lyophilised compositions of the present invention, particularly compositions containing ammonium sulphate as the stabilising salt and Trehalose as the cryoprotectant are stable at ambient temperature and at 37° C. for 16 months.

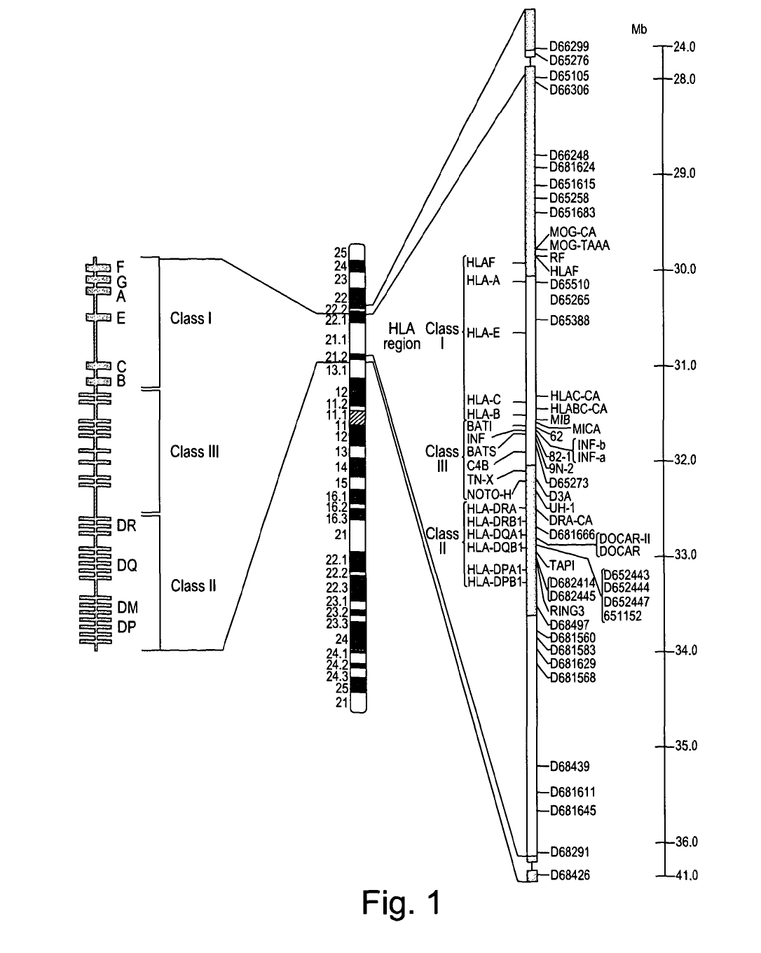

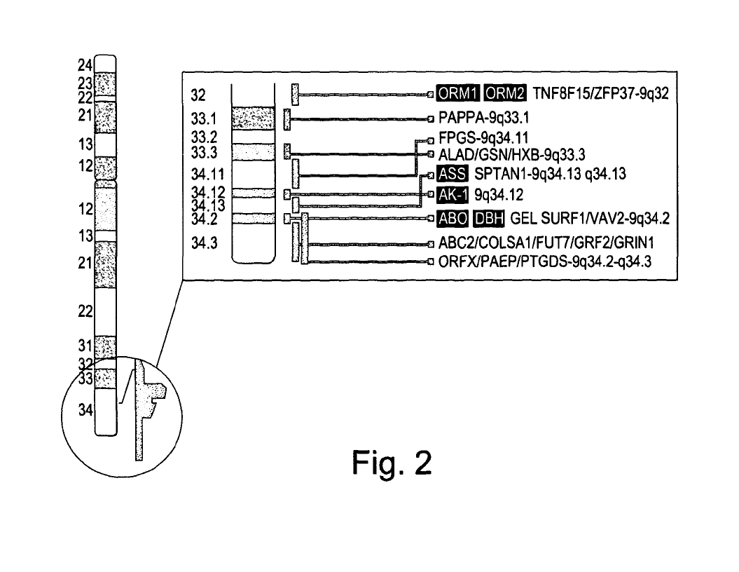

### DETAILED DESCRIPTION OF THE INVENTION

Accurate genotyping and more particularly HLA and/or ABO genotyping is complicated by the highly polymorphic nature of the respective genomic regions. For example, there are hundreds of different allele sequences at the various HLA class I and class II genes. Current methods, based on Sanger-sequencing technology, are limited in their ability to resolve “phase ambiguities” which occurs commonly when an individual is heterozygous at many positions that are very close to one another. The challenge, therefore, is to determine which polymorphic sequences go together with which alleles

In determining the type of assays to use, the level of typing resolution needed must be considered. For solid organ transplantation, resolution at an antigen level may be sufficient for HLA-A,B, Cw, DR and DQ antigens. However, allele level typing may be needed in cases where the patient and donor appear to share an antigen but have different alleles of that antigen and the patient has antibody to the donor's allele.

HLA nomenclature distinguishes antigens and alleles. Letters denote the HLA locus involved, e.g., HLA-A,B,Cw,DRB1, DRB3-5, DQB1. Numbers stand for either antigens or alleles. Antigens were defined first, based on their serologic reactivity. As serologic definition of HLA antigens improved, it was recognized that some antigens could be divided into subtypes or “splits” like the division of blood group A into A1 and A2. These “splits” share epitopes which result in serologic crossreactivity. Antigens that share serologic epitopes are part of a CrossReactive Group of antigens or CREG. Molecular techniques define HLA based on DNA sequences, allowing the differentiation of HLA alleles, some of which differed by only one nucleotide.

Rules for naming HLA antigens and alleles are established by a WHO Nomenclature Committee (Marsh S G, et al. Human Immunol. 2005; 66:571). HLA alleles are designated by the locus followed by an asterisk and a numeric allele designation. HLA alleles minimally have four digits, with the first two denoting the serologic equivalent if it is known (Schreuder G M, et al. Human Immunol. 2005; 66:170). The third and fourth digits specify the allele. Additional digits are added to indicate synonymous substitutions (no change in protein sequence), null alleles and other special cases. It is important to note that there are groups of closely related alleles that can encode the same serologically defined antigen.

Hereditary hemochromatosis is a genetic disease with an estimated carrier frequency of 1 in 10 that would benefit from improved testing. Caused by a progressive iron overload, it is characterised by severe complications and a potentially lethal progression that could be entirely prevented with regular iron removal by means of bleeding. The HFE gene is located on short arm of chromosome 6 at location 6p21.3. Two mutations, C282Y and H63D, located respectively in exons 4 and 2, impair HFE function and are frequently tested for, along with a third variant located on exon 2 (S65C). Two genotypes (C282Y homozygote and compound heterozygote C282Y/H63D) are associated with risk of HC.

Although almost any tissue source can be used for molecular genotyping, lymphocytes from peripheral blood, for example, are most often used. It is also possible to utilise samples obtained through non-invasive means, for example by way of cheek swab or saliva-based DNA collection. Various suitable methods for extracting DNA from such sources are known in the art. These range from organic solvent extraction to absorption onto silica coated beads and anion exchange columns. Automated systems for DNA extraction are also available commercially and may provide good quality, high purity DNA.

All molecular HLA typing methods rely on amplifying sufficient copies of HLA sequences by the polymerase chain reaction (PCR). PCR amplification for HLA typing can be locus-, allele group-, or allele-specific depending on the technique in use. The PCR amplicons are further tested to detect specific polymorphic sequences, such as those that define specific HLA alleles or groups of related alleles that encode a specific HLA antigen.

Polymorphism in HLA molecules occurs largely in the protein domains that comprise the peptide binding regions. For HLA class I molecules, DNA typing methods focus on exons 2 and 3, which encode the 1 and 2 domains of the HLA-A, B, and Cw heavy chains. For class II molecules, the peptide binding site is comprised of the first domains of the and, chains which are encoded by exon 2 of their respective genes.

For HLA-DR, only the beta chain is polymorphic. Therefore, DR typing schemes generally concentrate on exon 2 of the DRB1, DRB3, DRB4, and DRB5 genes. It should be noted that alleles of the DRB1 locus encode the serologically defined DR antigens 1-18, while the DRB3-5 loci encode DR52, DR53, and DR51 antigens, respectively. All individuals carry the DRB1 gene, but the presence of the DRB3-5 genes varies with specific haplotypes carrying different DRB1 genes. Therefore, individuals can have as few as one DR antigen (if they are homozygous for DR1, 8 or 10) or as many as four DR antigens (e.g., DR15, DR17, DR51, DR52). Additional attention has focused on HLA-DQ and DP antigens in recent years, due to reports of HLA antibodies specific for both alpha and beta chains. Consequently, complete typing for HLA-DQ and DP must also consider exon 2 of the polymorphic alpha chain.

Typing ambiguities, or multiple possible allele assignments, are encountered with all molecular typing methods and result from the degree of shared sequences among HLA alleles as well as the sheer number of recognized HLA alleles. Ambiguities can arise when two or more alleles have the same sequence in the regions tested and cannot be differentiated without further testing. Ambiguous heterozygous combinations occur when two alleles at a locus are analyzed and the polymorphic sequences analyzed can be accounted for by two or more different combinations of known alleles.

Thus, there is a need for new compositions that enable these problems to be overcome.

Surprisingly, there are no products available that possess all of the components required for a genotyping reaction in a single composition. This means that laboratory technicians have to perform many additional cumbersome and time-consuming steps before a genotyping reaction is ready to be performed. The inclusion of such steps, which may include pipetting and dilution steps, means that the efficiency and accuracy of the genotyping reactions is reduced. For example, problems with primer dilutions may mean that a PCR reaction fails, either by inclusion of excess primers or by neglecting to add primers to the reaction. In addition, in order to obtain all of the information required to ascertain the genotype of a patient, organ or clinical sample, usually many individual amplification reactions are necessary. When clinical samples are in short supply or when patients are awaiting donors/transplants, such delays may prove costly both financially in terms of patient mortality. The inventors have now developed a number of genotyping compositions that overcome many problems associated with currently available compositions and methods.

Genotyping compositions of the present invention are for use in a reaction for the amplification and/or synthesis of at least one polynucleotide.

The term “amplification and/or synthesis” as used herein is intended to mean the process of increasing the numbers of a template polynucleotide sequence by producing copies. Accordingly it will be clear that the amplification process can be either exponential or linear. In exponential amplification the number of copies made of the template polynucleotide sequence increases at an exponential rate. For example, in an ideal PCR reaction with 30 cycles, 2 copies of template DNA will yield 230 or 1,073,741,824 copies. In linear amplification the number of copies made of the template polynucleotide sequences increases at a linear rate. For example, in an ideal 4-hour linear amplification reaction whose copying rate is 2000 copies per minute, one molecule of template DNA will yield 480,000 copies.

Whilst many methods of amplifying or synthesising polynucleotides are known in the art, generally genotyping compositions of the present invention are for use in the Polymerase Chain Reaction (PCR).

As used herein, the term ‘polynucleotide’ refers to deoxyribonucleic acid (DNA), but where appropriate the skilled artisan will recognise that the method may also be applied to ribonucleic acid (RNA). The terms should be understood to include, as equivalent, analogues of either DNA or RNA made from nucleotide analogues and to be applicable to single stranded (such as sense or antisense) and double stranded polynucleotides. The term as used herein also encompasses cDNA, that is complementary or copy DNA produced from an RNA template, for example by the action of reverse transcriptase.

The polynucleotide molecules may originate in double-stranded DNA (dsDNA) form (e.g. genomic DNA, PCR and amplification products and the like) or may have originated in single stranded form as DNA or RNA and may be converted to dsDNA form and vice-versa. The precise sequence of the polynucleotide molecules is generally not material to the invention and may be known or unknown.

In a particular embodiment the polynucleotide molecules are DNA molecules. More particularly the polynucleotide molecules are amplified from a primary sample which comprises the entire genetic complement of an organism, for example genomic DNA molecules including both intron and exon sequence (coding sequence), as well as non-coding regulatory sequences such as promoter and enhancer sequences. It could also be envisaged that polynucleotide sequences may be amplified from isolated fragments of genomic DNA such as particular chromosomes for example.

Compositions of the present invention will comprise at least one enzyme suitable for amplifying or synthesising a polynucleotide molecule or sequence.

Examples of enzymes with polymerase activity which can be used in the present invention are DNA polymerase (Klenow fragment, T4 DNA polymerase), heat-stable DNA polymerases from a variety of thermostable bacteria (such as Taq, VENT, Pfu, Tfl DNA polymerases) as well as their genetically modified derivatives (TaqGold, VENTexo, Pfu exo). A combination of RNA polymerase and reverse transcriptase could also be used to generate polynucleotide sequence. Particularly the enzyme has strand displacement activity, more particularly the enzyme will be active at a pH of about 7 to about 9, particularly pH 7.9 to pH 8.8, yet more particularly the enzymes are Bst or Klenow. Particularly the enzyme is a Taq DNA polymerase enzyme. Compositions of the present invention may include between 0.1 units and 2 units of polymerase enzyme, more particularly 0.1 and 1 units, yet more particularly 0.1, 0.2, 0.3, 0.4, 0.5, 0.6, 0.7, 0.8, 0.9 units or any range therebetween.

In certain embodiments the composition may incorporate systems or components that inhibit polymerase activity at ambient temperature. For example, such inhibitors may include antibodies or covalently bound inhibitors that only dissociate after a high-temperature activation step. Compositions may also incorporate hybrid polymerases that are inactive at ambient temperature but which are instantly activated at elongation temperature.

It will be apparent to one skilled in the art that in a polynucleotide amplification or synthesis reaction it will be necessary to include 5′-triphosphates (NTPs). The nucleotides may be nucleoside triphosphate molecules in the form of deoxyribonucleotide triphosphates, for example dATP, dTTP, dCTP, dGTP, dUTP, or are ribonucleoside triphosphates for example ATP, UTP, CTP, GTP. The nucleoside triphosphate molecules may be naturally or non-naturally occurring. In preferred embodiments, NTPs are modified NTPs having a 3′-substitution. Preferably such a 3′-substitution will impair or prevent polymerase mediated oligonucleotide primer extension prior to the initial incubation period at an elevated temperature of nucleic acid replication, such as in the initial denaturation step of PCR. Thus, preferably the 3′-substitution group of the NTPs is a ‘blocking group’ that converts to an open 3′-hydroxyl (3′-OH) group during or after the initial denaturation step of the nucleic acid replication and, where applicable, during subsequent replication cycles.

The concentration of dNTPs generally refers to the concentration of each dNTP: that is, the given concentration refers to the concentration of each nucleotide type in the composition, for example, dATP, dTTP, dCTP, dGTP or dUTP, not the total concentration of all dNTPs combined. By way of non-limiting example, this would mean that a 2.5 mM dNTP mix for PCR contains 2.5 mM of EACH dNTP, and 10 mM TOTAL dNTPs. Suitable concentrations of dNTP are from about 0.1 mM to about 0.3 mM, particularly 0.2 mM to 0.3 mM, yet more particularly 0.15 mM, 0.2 mM, 0.25 mM, 0.3 mM or any range therebetween.

Suitable thermolabile protecting groups have been described in literature for use in the polynucleotide synthesis processes, for example in WO/2009/151921. These have not previously been utilised in the field of genotyping.

Preferably the compositions incorporate at least one cryoprotectant. The term “cryoprotectant” refers to a compound or composition that can protect the activity of a biologically active molecule or a reagent during freezing, drying, and/or reconstitution of the dried substance. In the context of the present invention the term refers to a substance that, when included in aqueous solutions, protects enzymes dissolved in the aqueous solutions from loss of enzymatic activity due to drying or freezing of the aqueous solutions. The term “lyophilised” or “lyophilisation” refers to drying a substance by freezing it in a high vacuum or to removing water from a frozen substance by sublimation under lowered pressure. Examples of cryoprotectants include carbohydrates and polyols, such as trehalose, glucose, sucrose, glycerol, polyethylene glycol, and sorbitol. A preferred cryoprotectant is trehalose, more particularly trehalose di-hydrate. Particularly the at least one cryoprotectant is present at between about 0.05% w/v to about 10% w/v, more particularly between about 2% w/v and 7% w/v, yet more particularly at about 2%, 3%, 4%, 4.5%, 5%, 5.5%, 6%, 7% w/v or any range therebetween.

In contrast to U.S. Pat. No. 6,153,412 in which it is stated that ammonium sulphate may be disadvantageous in PCR, particularly lyophilised PCR compositions, the inventors have discovered that the use of ammonium sulphate, (NH₄)₂SO₄ (CAS 7783-20-2) is beneficial when incorporated into lyophilised PCR compositions of the present invention, including the genotyping compositions of the present invention. The Inventors have also discovered that ammonium sulphate has further advantages in genotyping particularly for amplifying across introns. Ammonium sulphate may be included in the compositions, particularly lyophilised compositions, at concentrations above those used in a genotyping reaction and therefore requiring dilution. Thus, a lyophilised composition may be rehydrated with an aqueous sample, particularly an aqueous sample comprising a polynucleotide template, sufficient to dilute a reagent present in 10×, 9×, 8×, 7×, 6× or 5× concentrated form to 1× concentration ready for use. For a lyophilised composition a 10× concentrated amount of ammonium sulphate is preferably between 0.05M and 2M, more particularly between 0.1M and 0.3M, more particularly 0.2M. Further particular concentrations include 0.15M, 0.16M, 0.17M, 0.18M, 0.19M, 0.2M, 0.21M, 0.22M, 0.23M, 0.24M, 0.25M or any range therebetween.

Compositions of the present invention may optionally comprise one or more buffering agents. The term “buffering agent” refers to a reagent that can reduce changes to the concentration of free hydrogen ions in a solution, and thus can maintain a particular pH or pH range. Again, such buffering agents may be present in concentrated form, for example 10×, 9×, 8×, 7×, 6× or 5×. Suitable concentrated amounts are between 0.05M and 2M, more particularly between 0.1M and 0.7M, more particularly between 0.4M and 0.7M and yet more particularly 0.45M, 0.55M, 0.6M, 0.65M, 0.66M, 0.67M, 0.68M, 0.69M, 0.7M, 0.75M or any range therebetween. Alternatively amounts of such agents may be measured in terms of weight/volume percentage. Suitable amounts include between 0.1% and 10%, more particularly between 0.2% and 7% for example, more particularly 0.3%, 0.35%, 0.4%, 0.45%, 0.5%, 0.55%, 0.6%, 0.65%, 0.7%, 0.75%, 0.8%, 0.85%, 0.9%, 0.95%, 1% or any range therebetween. Suitable buffering agents include Tris-HCl, more particularly 0.67M Tris-HCl, yet more particularly 0.67M Tris-HCl, pH8.9. Other suitable buffering agents may include detergents such as TWEEN®, more particularly 0.5% TWEEN®. Other suitable buffering agents and concentrations will be apparent to one skilled in the art.

Compositions of the present invention may include other optional components such as bulking agents, salts, oils, dyes or waxes. A particular bulking agent is carbowax20M which may be present at between 0.05% and 2%, more particularly 1% w/v. Particular dyes include patent blue and/or cresol red, for example at levels of between 0.05% and 1%, for example 0.08% w/v.

In certain embodiments the compositions of the present invention comprise at least one genotyping primer that comprises a template binding region and a tail region.

A primer generally is a ribo- or deoxyribo-polynucleotide, which is usually single stranded, may be naturally occurring or synthetic, and usually include a sequence of between about 5 to about 50 nucleotides, more preferably about 10 to about 30 nucleotides or more preferably about 15 to about 25 nucleotides. Oligonucleotides may contain one or more modification groups. Oligonucleotides may include DNA, RNA, PNA, LNA, and/or other modified nucleosides.

The genotyping primer(s) or diagnostic primer(s) of the present invention incorporate a diagnostic portion that it is substantially complementary to a diagnostic portion of the polynucleotide to be amplified and includes a target base sequence. Under hybridising conditions, the diagnostic portion of the primer will hybridise to the diagnostic portion of the polynucleotide to be amplified. The nucleotide sequence of the at least one diagnostic or genotyping primer is such that the 5′ or 3′ terminal nucleotide of the diagnostic primer is complementary either to a suspected variant nucleotide (such as a single nucleotide polymorphism) or to the corresponding normal nucleotide. Thus, during PCR, for example, an extension product of the diagnostic primer will be synthesised when the terminal nucleotide of the diagnostic primer is complementary to the corresponding nucleotide in the target base sequence but no extension product will be synthesised when the said terminal nucleotide of the diagnostic primer is not complementary to the corresponding nucleotide in the target base sequence.

In order to generate an amplification product, generally it will be necessary for any reaction to comprise both forward and reverse primers. Thus, compositions may comprise both at least one genotyping or diagnostic primer (which may be either the forward or reverse primer) and at least one amplification primer (which may be the other of the forward or reverse primer).

The forward and reverse primers may be of significantly different lengths; for example one may be 20-40 bases, and one may be 40-100 bases in length. The nucleotide sequences of the forward and reverse primers are selected to achieve specific hybridisation to the sequences to be amplified under the conditions of the annealing steps of the amplification reaction, whilst minimising non-specific hybridisation to any other sequences present. Skilled readers will appreciate that it is not strictly required for the primer-binding sequence to be 100% complementary, a satisfactory level of specific annealing can be achieved with less than perfectly complementary sequences. In particular, one or two mis-matches in the adaptor-target specific portion can usually be tolerated without adversely affecting specificity for the template.

As discussed above, genotyping primer(s) or diagnostic primer(s) of the present invention may further incorporate a tail region. In certain embodiments the tail region increases the size of the primer by between 10 and 200 base pairs, more particularly by 10, 20, 30, 40, 50, 60, 70, 80, 90, 100, 110, 120, 130, 140, 150, 160, 170, 180, 190, 200 or any range therebetween. In this case, it is important that the tail region is not complementary to either the amplification primer or the target polynucleotide sequence. Thus, the tail region is an ‘unmatched region’ which exhibits a sufficient degree of non-complementarity such that it is not capable of fully annealing to any other components of the amplification reaction under standard annealing conditions for a primer extension or PCR reaction.

In other embodiments the tail region is not a nucleotide sequence but is a moiety that increases the molecular weight of the primer causing the primer or amplicon comprising it to appear, for example on a gel, to be of greater nucleotide length than it actually is. Suitable moieties include amine groups, biotin or spacers made of polyethylene glycol or other carbon containing compounds for example C6, C7, C8, C9, C10, C11 or C12 spacers.

In yet other embodiments the tail region is a positively charged moiety. In still yet other embodiments the tail region is a negatively charged moiety. The use of charged moieties to alter the net charge of a PCR amplicon may also increase or decrease the migration of a PCR amplicon through, for example, a gel matrix. Such an increased or decreased charge enables the amplicon to appear to comprise a greater or lesser number of nucleotides. This enables better differentiation or discrimination between amplicons particularly on the basis of size/length, more particularly when visualised using size separation techniques such as gels, gel electrophoresis, capillary electrophoresis and the like.

An important feature of the invention is that the tail region is inert, serving only to increase the size, or perceived size, of one or more amplicons in a reaction mixture. The tail region may be copied and amplified as a ‘consequence’ of the PCR reaction in the 5′ to 3′ direction. However, whilst it may bind to its complementary copy it generally does not bind directly to other components of the PCR reaction such as specific primers, oligonucleotide probes or nested primers. Generally the compositions and methods of the invention are for use in an amplification reaction that is performed once—i.e. there is no need for subsequent amplification or PCR reactions utilising the products of the amplification. Thus, once the amplification has been performed, the products of that amplification reaction may be analysed directly and may be visualised by methods known in the art such as gel electrophoresis or capillary electrophoresis. Many amplification methods of the prior art require further amplification steps to be performed, for example using further primers such as nested primers or specific probes. Thus, the compositions and methods of the present invention provide significant advantages.

The genotyping or diagnostic primer(s) may be used to artificially elongate the primer specifically to make it seem as if two or more cis located polymorphisms are further apart than they biologically are. The differentially sized amplicons can be used to discriminate between two single nucleotide polymorphisms located at the same SNP site (+/−10 bp) using (for example) a universal forward amplification primer and two reverse genotyping primers for the SNPs whereby the two reverse primers are distinguished by the 3′ mismatch but also the tail which serves to artificially lengthen the primer(s) and subsequently the amplicon(s) produced therefrom.

Discrimination of the two SNP's can then be performed using slab gel electrophoresis for example or capillary type electrophoresis. An advantage of using these diagnostic primers of the present invention is that they can be applied to complex genotyping such as HLA. The invention enables one skilled in the art to condense genotyping reactions, such as those used in SSP or ARMS from, for example, 24 reactions for a DR genotype to, for example, 7 reactions for a DR type by applying a multiplexed strategy with extended primers to help discriminate between amplicons.

The technique is well suited to generating ‘single tube assays’ whereby a collection of SNPs is multiplexed within a single reaction rather than multiple reactions, for example, in HFE typing where there are 5 well known SNPs that define the common forms of haemachromatosis. The primers/technique may be applied to conventional SSP whereby the problem is that the two polymorphisms to be identified are closely located so that a conventional SSP type reaction would produce an amplicon of less than 120 bp. These reaction amplicons are difficult to distinguish from PCR artefacts such as primer-dimers. Use of the composition and primers of the invention can be used to make this SSP reaction brighter on the gel and more distinguishable from normal SSP.

Preferably composition(s) of the present invention is/are lyophilised compositions. Lyophilised compositions of the present invention may be prepared by freeze-drying aqueous reaction mixtures comprising the various components such as polymerase enzyme, dNTPs, cryoprotectant, ammonium sulphate, primers, buffering agents and the like. Briefly, the aqueous reaction mixture is prepared and liquid removed to provide a concentrated reaction mixture that is substantially lacking in water, i.e. is lyophilised. A lyophilised reaction mixture simplifies the process of preparing amplification reaction mixtures—all the necessary components, with the exception of template DNA and water are present. Lyophilisation also means that the compositions may be kept at room temperature rather than under special storage conditions such as in a fridge or freezer.

Lyophilisation (also known as freeze-drying) is an effective process for preserving biological reagents without loss of activity of the biological reagent. Lyophilisation involves removing water content by sublimation from a frozen mixture, usually under vacuum. PCR is a widely used molecular biology procedure in diagnostic and molecular analysis of nucleic acids such as DNA and RNA. Lyophilised reagents for PCR that include all of the ingredients for PCR except template DNA are especially useful for diagnostic kits because of the stability of the kit and the ease of use of the kit: all the consumer has to do is add DNA to the lyophilised PCR and proceed with the amplification process.

In addition, the lyophilised compositions of the present invention are room temperature stable for at least 12 months. Room temperature stable, means that the compositions are stable without refrigeration, they have been shown to be stable at temperatures of up to 37 C for at least 12 months. This has great advantages in terms of reagent storage and transport.

Not all PCR templates amplify with equal efficacy; it is well known that templates with a high GC content, or poly GC regions are difficult to amplify compared to non GC regions. GC-rich regions of the genome include important regulatory domains including promoters, enhancers, and control elements consist of GC-rich cis-elements (Wilson et al., 1997). Most housekeeping genes, tumor-suppressor genes, and approximately 40% of tissue-specific genes contain high-GC sequences in their promoter region, making their DNA less amenable to amplification. PCR across HLA introns is useful for PCR-SSP immunogentics genotyping (Bunce et al Tissue Antigens. 1995 November; 46(5):355-67) but amplification across these introns can be difficult due to GC rich sequences that can cause amplification failure and false negative reactions can occur. To overcome the problems associated with the amplification of GC-rich genes (and/or using GC-rich primers), several approaches have been developed. Organic molecules such as dimethyl sulfoxide (DMSO), glycerol, polyethylene glycol, formamide, betaine, 7-deaza-dGTP, and dUTP have been included in the reaction mixture and have been shown to improve the amplification of GC-richDNA sequences (Baskaran et al., 1996; Chakrabarti and Schutt, 2001; Henke et al., 1997; Kang et al., 2005; Musso et al., 2006; Mutter and Boynton, 1995; Pomp and Medrano, 1991; Sidhu et al 11996; Sun et al., 1993; Turner and Jenkins, 1995; Weissensteiner and Lanchbury, 1996). The problem for lyophilised PCR reactions is that organic solvents and many other additives that are beneficial in PCR do not lyophilise effectively resulting in partially sublimated reagents that rapidly loose activity, are unstable and ineffective for PCR. The inventors have discovered a unique combination of components that utilises ammonium sulphate to allow the amplification of difficult templates and thus enable the use of freeze dried reaction mixtures for multiple allele-specific amplification reactions such as those used in ARMS or SSP type applications, in particular genotyping, more particularly HLA genotyping and/or ABO genotyping and HFE genotyping.

Preferably the genotyping composition is a multiplex genotyping composition. Multiplex means that the composition consists of multiple primer sets within a single reaction mixture to produce amplicons of varying sizes that are specific to different DNA sequences. By targeting multiple genes at once, additional information may be gained from a single test run that otherwise would require several times the reagents and more time to perform. The use of genotyping primers of the present invention permits manipulation of amplicon sizes, for example their base pair length, such that amplicons relating to different alleles which would ordinarily be of similar sizes are sufficiently separated to form distinct bands such as when visualized by gel electrophoresis. Such enhanced separation of bands leads to significant gains in the efficiency of genotyping.

Allele specific PCR (such as ASA, ARMS, SSP) is a useful technique in genotyping as it allows the detection of polymorphisms in a cis-located method. The method has been popularised as a rapid and relatively easy method of genotyping by numerous applications particularly in the field of HLA genotyping and other complex genotyping applications such as ABO genotyping. Allele-specific PCR is currently only useful when applied to polymorphisms that are located at least 80 nucleotides apart, up to a maximum of 1000 bp apart. The problem in identifying SNPS that are closer than 80 bp apart is that the PCR amplicon is too small to be readily identified by standard gel electrophoresis as the amplified PCR fragment. To allow the detection of closely linked polymorphisms in a reliable and predictable manner the Inventors have utilised primers with a wholly or partially mismatched 5′ extension that effectively increases the length of a primer from the standard 16-25 bases to a 50 bp primers that does not affect the specificity of the reaction. The extra 25 bases on each primer adds 50 bp to the PCR amplicon which improves the identification of small PCR fragments. As stated above, the use of artificially extended PCR fragments is even more useful when applied to multiplexed PCR as by using differentially extended primers for different polymorphisms located at the same position allows for different size amplicons to be generated for different polymorphisms at the same position. The artificial amplicon extension allows the use of differentially extended primers to be used in PCR reactions to artificially extend PCR amplicon sizes. This benefits genotyping methods via direct PCR analysis by allowing improved identification of single nucleotide polymorphisms where the distance between the SNPs is too close for conventional PCR, and secondly allows multiplex identification of SNPs where the SNPs of different alleles are at the same position on various different alleles. The normal part of the primer substantially matches the target DNA, whereas the extended part of the PCR primer is designed so that it does not substantially match the flanking target DNA.

Genotyping compositions of the present invention may comprise at least 2, 3, 4, 5, 6, 7, 8, 9, 10, 11, 12, 13, 14, 15, 16, 17, 18, 19, 20 or greater primers ore forward/reverse primer pairs. Particularly such primers are for amplifying an HLA gene and/or ABO gene. In particular embodiments the HLA genes are HLA-A, HLA-B, HLA-C, HLA-DRB1, DRB3, DRB4, DRB5, DQB1, DQA1, DPA1, DPB1 or any of the polymorphic genes in the MHC cluster on chromosome 6.

Example primer sequences are provided in the experimental section below however, it will be apparent to one skilled in the art that artificial amplicon extension may be utilised to incorporate a tail into any primer sets currently known or that are being developed to a particular sequence or SNP.

Compositions of the present invention may be rehydrated and/or diluted prior to use. Particularly such rehydration is by addition of a solution of template polynucleotide(s). For example, a solution comprising between 0.001 and 1000 pg of DNA or any value therebetween. Alternatively the rehydration is by addition of water or a buffer solution and template polynucleotide is added separately. The addition of an aqueous solution rehydrates lyophilised compositions of the invention and dilutes the components of that composition (which will generally be in concentrated form) to their working concentration.

The methods of the invention are similar to those developed in relation to ARMS technology disclosed in European Patent No. 0332435 and U.S. Pat. No. 5,595,890 for example, the disclosure of which is herein incorporated by reference. ARMS is a trademark of ZENECA Limited.

Briefly, the method comprises detecting the presence or absence of at least one variant nucleotide in one or more nucleic acids contained in an aqueous sample, comprising, treating the aqueous sample with appropriate nucleoside triphosphates, a Taq DNA polymerase and at least one genotyping or diagnostic primer, the nucleotide sequence of the at least one diagnostic primer being such that it is substantially complementary to the diagnostic portion, the 5′ or 3′ terminal nucleotide of the diagnostic primer being either complementary to the suspected variant nucleotide or to the corresponding normal nucleotide, whereby an extension product of the diagnostic primer is synthesised when the said terminal nucleotide of the diagnostic primer is complementary to the corresponding nucleotide in the target base sequence, no extension product being synthesised when the said terminal nucleotide of the diagnostic primer is not complementary to the corresponding nucleotide in the target base sequence, and detecting the presence or absence of the suspected variant nucleotide from the presence or absence of an extension product. However, in the present invention the method is characterised in that all of the components of the reaction, more specifically the nucleoside triphosphates, Taq DNA polymerase and primers are in lyophilised form and/or the diagnostic primer comprises a tail region that increases the size of an extension product synthesised but which is non-complementary to the nucleotide sequence.

The method of the invention described above may be further refined as including the following steps: (i) treating the sample with appropriate nucleoside triphosphates, a Taq DNA polymerase, a diagnostic primer for a diagnostic portion of a target base sequence and a corresponding amplification primer under hybridising conditions, the nucleotide sequence of the said diagnostic primer being such that it is substantially complementary to the said diagnostic portion, the 5′ or 3′ terminal nucleotide of the diagnostic primer being either complementary to the suspected variant nucleotide or to the corresponding normal nucleotide, whereby an extension product of the diagnostic primer is synthesised when the said terminal nucleotide of the diagnostic primer is complementary to the corresponding nucleotide in the target base sequence, no extension product being synthesised when the said terminal nucleotide of the diagnostic primer is not complementary to the corresponding nucleotide in the target base sequence, any extension product of the diagnostic primer formed being capable of serving as a template for synthesis of an extension product of the said amplification primer after separation from its complement, (ii) treating the sample under denaturing conditions to separate the primer extension product from as template where such extension product is formed, (iii) contacting single strands produced in step (ii) with appropriate nucleoside triphosphates, a Taq DNA polymerase, a diagnostic primer and an amplification primer as herein defined whereby, where possible, to synthesise further extension products using the single strands produced In step (ii) as templates (iv) repeating steps (ii) and (iii) a sufficient number of times to result in detectable amplification of the appropriate nucleotide sequence, and (v) detecting the presence or absence of the suspected variant nucleotide from the presence or absence of an amplification product obtained in step (iv). The method is again characterised in that all of the components of the reaction, and specifically the nucleoside triphosphates, Taq DNA polymerase and diagnostic primers are in lyophilised form and the diagnostic primer comprises a tail region that increases the size of an extension product synthesised but which is non-complementary to the nucleotide sequence.

Particularly the tail region of the at least one genotyping or diagnostic primer is at the 5′ end of the diagnostic primer and wherein the terminal nucleotide of the diagnostic primer being either complementary to the suspected variant nucleotide or to the corresponding normal nucleotide is at the 3′ end of the diagnostic primer.

The use of lyophilised compositions means that HLA and/or ABO and/or HFE genotyping kit(s) may be easily and conveniently provided. Such kits may comprise:
(i) a Taq DNA polymerase enzyme;
(ii) at least one nucleotide selected from the group consisting of dATP, dCTP, dTTP, dGTP and dUTP;
(iii) a cryprotectant;
(iv) ammonium sulphate
(v) at least one genotyping primer; and
(vi) optionally a buffering agent.

Particularly the at least one primer comprises a template binding region and a tail region. Preferably at least one nucleotide comprises a thermally labile 3′-substitution group.

Such kits may include further components, for example, buffers, diluents, gels, detection reagents, fluorophores, dyes or stains, control DNA samples, gloves, pipette tips, instruction manuals and the like.

Such kits may be room temperature stable for at least 12 months.

### Experimental Overview

The following experimental details describe the complete exposition of one embodiment of the invention as described above.

### Example 1

### Preparation of Lyophilised Reaction Mixtures

To determine the effect of lyophilization on PCR, PCR reaction mixtures were prepared in order to give the following final (lyophilised concentrations):

10×PCR buffer—0.2M Ammonium sulphate; 0.67 Tris-HCl pH8.9; 0.5% TWEEN®
Bulking reagent—5% w/v Trehalose di hydrate; 1% Carbowax20M
dNTP—0.25 mM Bioline dNTP

### Dye—0.08 w/v Patent Blue/Cresol Red

Taq polymerase—0.5 units
Solutions were quick-frozen at −40° C. and freeze-dried using a lyophiliser (Ilshin Engineering, Korea). The following lyophilisation protocol was followed:
3 hours at −40° C. and atmospheric pressure
16 hours at −35° C. at <0.05 millibars
24 hours ramping to ambient temperature at <0.05 millibars

The resulting lyophilised PCR mixtures were reconstituted with distilled water and template—PCR reactions were carried out as described below.

### Example 2

### Multiplex PCR

It is useful to those skilled in the art of constructing SSP reactions for genotyping to be able to construct PCR reactions that amplify GC rich regions, in particular for HLA genotyping to enable the amplification to occur across introns, especially intron 2 of HLA class I sequences.

Example 2 demonstrates the use of multiplexed PCR using primers A1162, A1548 and A1159 at a final concentration of 0.3 uM. The combination of A1162 and A1548=547 bp, cross intron specific to B37s whilst the A1162 & A1159=146 bp exonic specific to B58s. DNA samples heterozygous for B*37 and B*58 and a homozygous B*37 sample were amplified with the aforementioned primers comparing a freeze dried potassium chloride buffer to freeze dried ammonium sulphate buffer. The ammonium sulphate buffer was constructed as described previously in this document and the KCL buffer was constructed by substituting ammonium sulphate for 500 mM potassium chloride. All PCR mixtures also had control amplification primers amplifying 1000 bp section of DRA gene to show that the PCR buffer was capable of amplification. Samples were amplified using standard amplification procedures in the following format
1. B37/B58 DNA with ammonium sulphate buffer
2. B58 DNA with ammonium sulphate buffer
3. B37/B58 DNA with KCl buffer
4. B58 DNA with KCl buffer

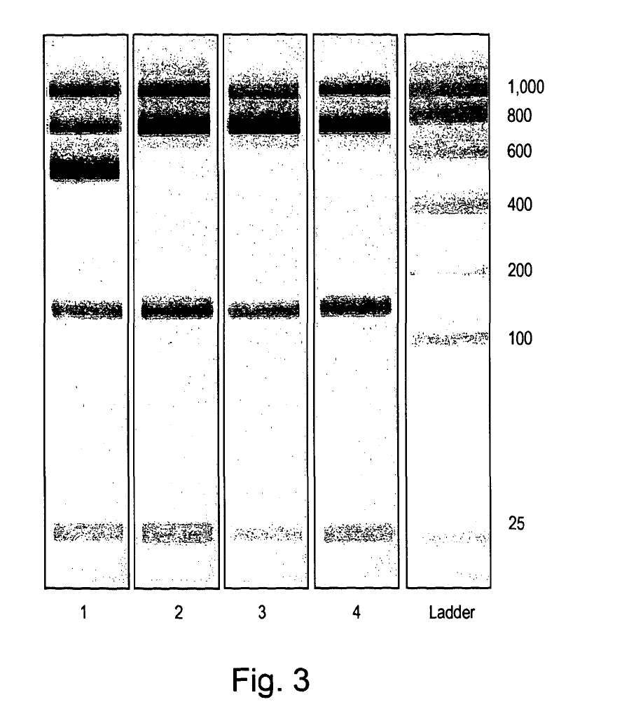

The samples were electrophoresed using lab901 screentape capillary electrophoresis system and the resulting images are displayed in FIG. 3 which compares amplification using ammonium sulphate buffer with potassium chloride based buffer.

The results show that only the ammonium sulphate buffer was competent in amplifying the 547 bp cross intron amplification. The KCl buffer was sufficient for amplifying the 1000 bp control and the short amplicon indicating the presence of B*58, but as can be seen in lane 3 the KCl buffer did not amplify the B*37 fragment as it was a cross intron amplification.

TABLE 1
Primer details.

| | | | 5′ | 5′ | |
|---|---|---|---|---|---|
| Reaction | | | concen- | customer | |
| name | 5′ ID | 5′ sequence | tration | info | |
| B66.1.1 | A1162 | GACGCCGCGAATCCGAGGAC | 0.3 uM | 206GAC | |
| B66.1.1 | A1162 | GACGCCGCGAATCCGAGGAC | 0.3 uM | 206GAC | |
| | | | 3′ | 3′ | |
| Reaction | | | concen- | customer | Intron |
| name | 3′ ID | 3′ sequence | tration | info | sizes |
| B66.1.1 | A1548 | CGGCCCCACGTCGCAACCAG | 0.3 uM | 368CTG | 348 |
| B66.1.1 | A1159 | ATACTATGACATACGACAGACGCCGCAGGTTCTCTCGGTAAGTCTACGC | 0.3 uM | 283GCG | 0 |

### Example 3

The following primer mixes were selected (all variants of the SSP DQA1*01 primers):

| Mix | Orientation | Name | Modification | Sequence |
|---|---|---|---|---|
| 1 | Sense | A000001 | | GAAGGAGACTGCCTGGCGGTG |
| | Antisense | A000003 | | TTTAATCATGATGTTCAAGTTGTGTTTTGC |
| 2 | Sense | A000005 | 5′ C12 amine | GAAGGAGACTGCCTGGCGGTG |
| | Antisense | A000007 | 5′ C12 amine | TTTAATCATGATGTTCAAGTTGTGTTTTGC |
| 3 | Sense | A000009 | 5′ C12 amine, | GAAGGAGACTGCCTGGCGGTG |
| | | | C18, C18 | |
| | Antisense | A000011 | 5′ C12 amine, | TTTAATCATGATGTTCAAGTTGTGTTTTGC |
| | | | C18, C18 | |
| 4 | Sense | A000063 | 5′ C12 amine, | TTTTTTTTTTGAAGGAGACTGCCTGGCGGTG |
| | | | C18 | |
| | Antisense | A000065 | 5′ C12 amine, | TTTTTTTTTTTTTAATCATGATGTTCAAGTTGTGTTTTGC |
| | | | C18 | |
| 5 | Sense | A000001 | | GAAGGAGACTGCCTGGCGGTG |
| | Antisense | A000011 | 5′ C12 amine, | TTTAATCATGATGTTCAAGTTGTGTTTTGC |
| | | | C18, C18 | |
| 6 | Sense | A000001 | | GAAGGAGACTGCCTGGCGGTG |
| | Antisense | A000065 | 5′ C12 amine, | TTTTTTTTTTTTTAATCATGATGTTCAAGTTGTGTTTTGC |
| | | | C18 | |
| 7 | Sense | A000009 | 5′ C12 amine, | GAAGGAGACTGCCTGGCGGTG |
| | | | C18, C18 | |
| | Antisense | A000003 | | TTTAATCATGATGTTCAAGTTGTGTTTTGC |
| 8 | Sense | A000063 | 5′ C12 amine, | TTTTTTTTTTGAAGGAGACTGCCTGGCGGTG |
| | | | C18 | |
| | Antisense | A000003 | | TTTAATCATGATGTTCAAGTTGTGTTTTGC |
| 9 | Sense | A000001 | | GAAGGAGACTGCCTGGCGGTG |
| | Sense | A000009 | 5′ C12 amine, | GAAGGAGACTGCCTGGCGGTG |
| | | | C18, C18 | |
| | Antisense | A000003 | | TTTAATCATGATGTTCAAGTTGTGTTTTGC |
| | Antisense | A000011 | 5′ C12 amine, | TTTAATCATGATGTTCAAGTTGTGTTTTGC |
| | | | C18, C18 | |
| 10 | Sense | A000001 | | GAAGGAGACTGCCTGGCGGTG |
| | Sense | A000063 | 5′ C12 amine, | TTTTTTTTTTGAAGGAGACTGCCTGGCGGTG |
| | | | C18 | |
| | Antisense | A000003 | | TTTAATCATGATGTTCAAGTTGTGTTTTGC |
| | Antisense | A000065 | 5′ C12 amine, | TTTTTTTTTTTTTAATCATGATGTTCAAGTTGTGTTTTGC |
| | | | C18 | |

Mixes were used to amplify DNA from International histocompatibility workshop (IHW) reference cell line KOSE (DQA1*0102) in standard 10 ul SSP reactions using Bioline Ammonium buffer and Biotaq. PCR protocol as follows:

| | | | | |
|---|---|---|---|---|
| Denature | 96 C. | 2 minutes | | |
| Denature | 96 C. | 15 seconds | | |
| | | | {close oversize brace} | 10 cycles |
| Anneal/extend | 65 C. | 60 seconds | | |
| Denature | 96 C. | 10 seconds | | |
| Anneal | 61 C. | 50 seconds | {close oversize brace} | 20 cycles |
| Extend | 72 C. | 30 seconds | | |
| Hold | 4 C. | | | |

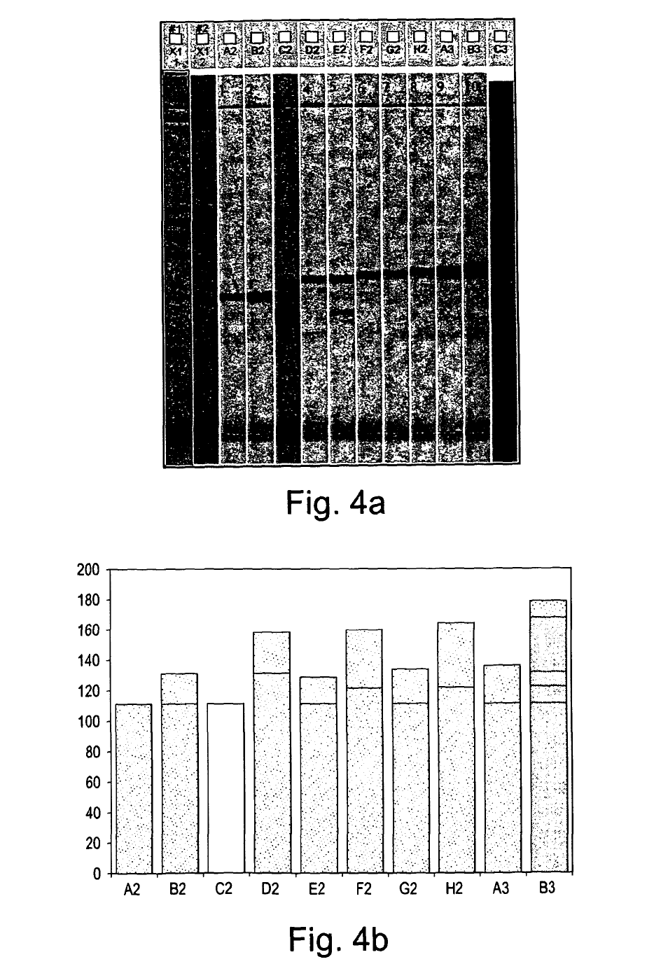

FIG. 4a shows a virtual image of the products of the reactions when amplicons were size separated on a Shimadzu MultiNA system.

### Results

Note that alternate lanes are from different chips and therefore can only be directly compared to each other (i.e. lane three should be compared to the ladder in lane 1 as they were both run on chip 1, and lane 4 should be compared to the ladder in lane 2 as they were both run on chip 2).

FIG. 4b is a graph showing the size of the peaks as given by the MultiNA. Note that these are normalised around the sizes given by the marker on the same chip each sample was run on, therefore alternate lanes CAN be directly compared. Blue (light gray) peaks show actual data recovered from the machine, whereas red (dark gray) peaks show expected sizes based ONLY ON SEQUENCE LENGTH, and do not take into account the effects of modification with amine or carbon spacers.

Information is tabulated below:

| | | Mod | Poly T | Poly T | Expected | Actual | |
|---|---|---|---|---|---|---|---|
| | Mod Sense | Antisense | Sense | Antisense | Size | Size | Difference |
| 1 | | | | | 111 | 110 | −1 |
| 2 | C12 Amine | C12 Amine | | | 111 | 131 | 30 |
| 3 | C12 Amine, | C12 Amine, | | | 111 | | |
| | C18, C18 | C18, C18 | | | | | |
| 4 | C12 Amine, | C12 Amine, | TTTTTTTTTT | TTTTTTTTTT | 131 | 158 | 27 |
| | C18 | C18 | | | | | |
| 5 | | C12 Amine, | | | 111 | 128 | 17 |
| | | C18, C18 | | | | | |
| 6 | | C12 Amine, | | TTTTTTTTTT | 121 | 159 | 38 |
| | | C18 | | | | | |
| 7 | C12 Amine, | | | | 111 | 133 | 22 |
| | C18, C18 | | | | | | |
| 8 | C12 Amine, | | TTTTTTTTTT | | 121 | 164 | 43 |
| | C18 | | | | | | |
| 9 | C12 Amine, | C12 Amine, | | | 111 | 135 | 24 |
| | C18 | C18 | | | | | |
| 10 | C12 Amine, | C12 Amine, | TTTTTTTTTT | TTTTTTTTTT | 111, 121, | 164, | 47 |
| | C18 | C18 | | | 131 | 178 | |

### Analysis and Conclusion

Lane C2 (sample 3) produced an aberant result and can be ignored.

Lane A2 (sample 1) should be considered the control—it is the basic amplification and the results show that the MultiNA recognises the size of the amplicons almost exactly (1 by difference). Inclusion of a 5′ C12 amine group artificially increases the amplicons by approximately 10 bp per modified primer (as in lane B2—sample 2). Addition of a C18 to this doesn't have much of an affect (as in sample 5 or 7). Sample 9 seems to suggest that the addition of C12 Amine and C18 to both primers actually reduces the distance traveled compared to just C12 amine. However, this may be because several bands are likely in this reaction, as in combinations of modified and unmodified primers. It is likely that the band given is a combination of four possible bands. This is supported by the results for sample 10 where three bands are to be expected due to the poly T on some primers, but only two are reported. This amplicon is slightly larger than the expected 121 by +C12 amine+C18 effect compared to sample 9.

In conclusion, the most precise way of artificially lengthening amplicons is to include polynucleotide bases at the 5′ end. C12 Amine and C18 give artificially larger products and this experiment demonstrates that both modification by polynucleotide and carbon spacers or amine are useful in multiplexing.

### Example 4

### Artificially Extended Primers

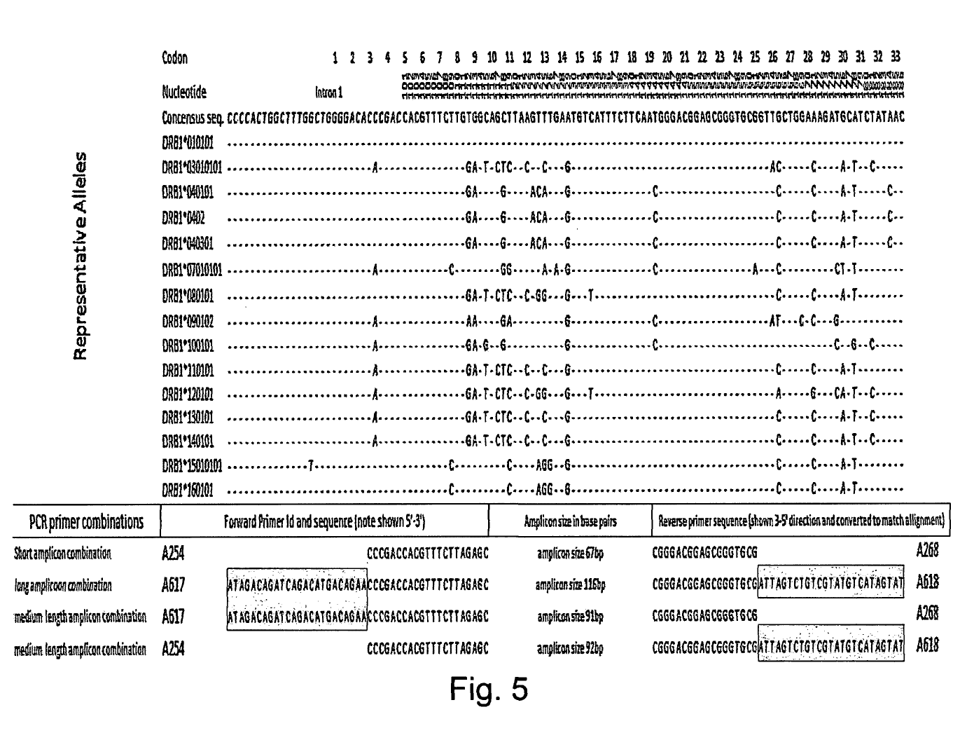

FIG. 5 illustrates the design, use and implementation of artificially extended primers to generate PCR amplicons that are visually improved to generate suitable genotyping primer combinations.

It is desirable in HLA genotyping to produce a single PCR reaction that amplifies all the known DR4 alleles in a single reaction without amplifying any non-DR4 alleles. Sequence alignments of all known (see http://www.ebi.ac.uk/imgt/hla/) alleles show that the only combination of polymorphisms that could be combined together to create this PCR would be the combination of the C115-C144 polymorphisms. Unfortunately these polymorphisms are too closely located so that normal primers such as A254 and A268 would produce a small amplicon of 67 bp that would be too short for routine clinical assessment in a panel of other HLA genotyping SSP reactions. A sub-100 bp amplicon is easily confused with primer-dimer artefacts such that there is an unacceptable risk that a positive reaction may be ignored by the observer. Secondly sub-100 bp amplicons take up little of the fluorochromic dye such as ethidium bromide and appear weak and fuzzy on gels. Thirdly the sub-100 bp amplicons migrate very fast on standard gel equipment and there is a risk that the amplicon may run into preceding or subsequent PCR lanes during electrophoresis.

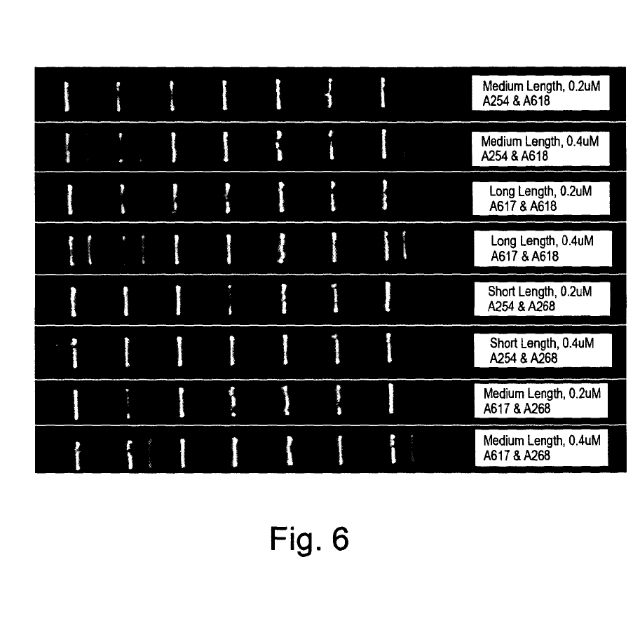

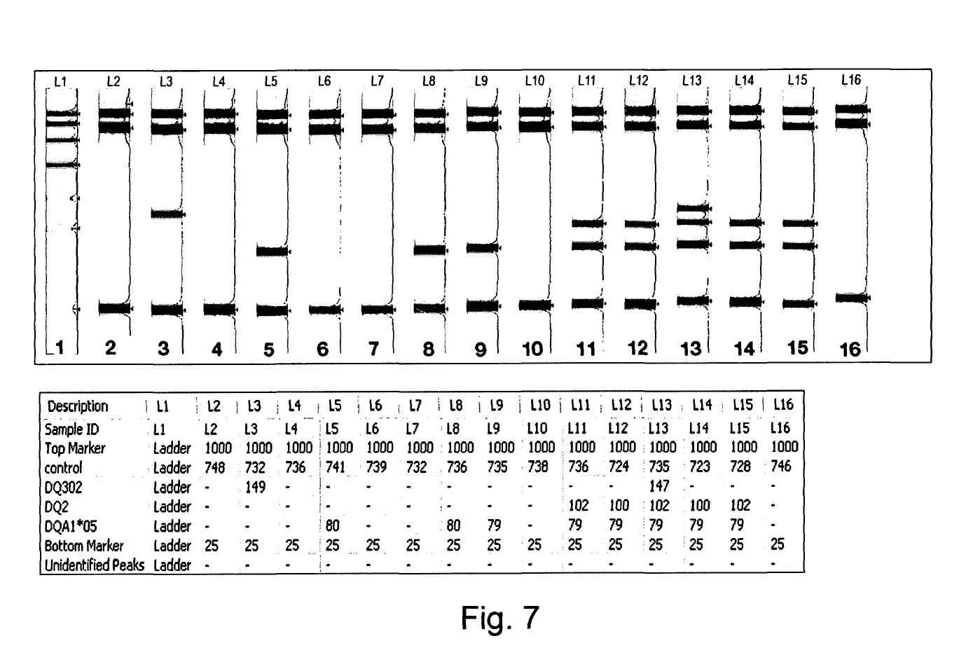

Primers A617 and A618 are artificially elongated primers designed to be substantially mismatched to the target beyond the usual active length of the PCR primer. The elongated tails are shown shaded. The sequences are selected to be substantially mismatched to target flanking regions and to minimise the interaction with each other to minimize the effect of primer-primer interaction which causes well known PCR primer-dimer effects or prevents the PCR from functioning at all. The combined length of the primers A617-A618 produce an amplicon 48% bigger than would otherwise be the case. As can be seen by the mobility in FIG. 6 the shorter amplicons have run into the next lane of the gel. Artificially lengthened amplicons are visibly stronger on the gel. This is useful as it allows both large and small amplicons to be run on the same gel with the same electrophoresis parameters.

### Example 5

Variably extended primers can be utilised in multiplexed PCR reactions to identify polymorphisms within 50 bp of each other that would be difficult to detect by standard multiplexed PCR. A multiplex PCR was created using primers 3-6: if some of the primers were not differentially extended the amplicons for alleles 1, 2 & 3 would all be the same size and thus not able to differentiate by any form of gel electrophoresis. Consequently to create allele-specific PCR reactions (SSP) for these alleles would require 4 separate PCR reactions. By using artificial extensions we were able to multiplex the primers into one PCR reaction where the amplicons identifying the various alleles can be identified and discriminated between as shown in the tables below.

| | |
|---|---|
| | 1 1 1 1 1 1 |
| | 1 2 3 4 5 6 7 8 9 9 |
| POSI- | 0 0 0 0 0 0 0 0 0 9 |
| TION | |
| Con- | ATACTAGCAGCAGCATCGACGACAAACACACATTTAGACG//ACGGCGCAGCAGCAGCAGACACGCAGACGCTCATCATCACGACGACACACAGC |
| sensus | |
| Allele 1 | -------------------------------G--------//----------------------------------------------------- |
| Allele 2 | -------------------------------G--------//----------T------------------------------------------ |
| Allele 3 | ----------------------------------------//----------T------------------------------------------ |
| Allele 4 | ----------------------------------------//----------------------------------------------------- |
| Primers | |
| | P3. 5-ATCGACGACAAACACACG-3 3-TCGTCGTCTGTGCGTCT-5 P5. |
| | P4. 5-(+bp tail)-ATCGACGACAAACACAC-3 3-ACGTCGTCTGTGCGTCT-(+60 bp tail)-5 P6. |
| Forward | Specitic for A41 |
| Primer 3 | |
| Forward | Specific for G41 |
| Primer 4 | |
| Reverse | Specific for A158 |
| Primer 5 | |
| Reverse | Specific for T158 |
| Primer 6 | |

| Forward | Reverse | | Alleleic | Extended | Size if not |
|---|---|---|---|---|---|
| Primer | primer | SNPs | Specificity | Amplicon size | extended |
| P3 | P5 | G41-A158 | Allele 1 | 152 bp | 152 bp |
| P3 | P6 | G41-T158 | Allele 2 | 212 bp | 152 bp |
| P4 | P5 | A41-A158 | Allele 4 | 182 bp | 152 bp |
| P4 | P6 | A41-T158 | Allele 3 | 242 bp | 152 bp |

Extended primers and the multiplexing method were applied to the detection of the alleles associated with Coeliac disease. Coeliac disease is associated with the HLA allele groups DQA1*05, DQB1*02 and DQB1*0302. Presence of these in cis or trans orientation can result in autoimmune response to gluten and consequently Coeliac disease. The aim of this variably extended PCR Multiplex experiment was to design a single PCR that identifies DQB1*02 and *0302 with DQA*05 capillary gel electrophoresis for the detection of the by fragments.

### Primers

Forward primers

| Primer | | | Primer |
|---|---|---|---|
| id | Position | Sequence | id |
| A558 | DQA 274 | TGCACTGACAAACATCGCTGTC | A556 |
| A550 | DQB 266 | ATAGACAGATCAGACATGACAGAAAACGCTGCTGGGGCTGCCTGC | A552 |
| | | | A551 |

Reverse primers

| | | Amp | |
|---|---|---|---|
| Position | Sequence | Size | Specificity |
| DQA 294 | GTAGAGTTGGAGCGTTTAATCAGAC | 67 bp | DQA1*05 group |
| DQB 317 | ATACTATGACATACGACAGACTAATTGTCTGCACACCGTGTCCAACT | 143 bp | DQB1*0302 group |
| DQB 508 | TGTCCACCGCCGCCCGTT | 105 bp | DQB1*02 group |

The above 5 primers were combined in a single freeze-dried PCR SSP reaction: primer pair A558-A556 amplify a 67 bp fragment of DQA. Primer A550 may combine with primer A552 to produce a 143 bp DQB1*0302 amplification or with A551 to produce a 105 bp DQB1*02 amplicon. The forward primer A550 was tailed so that combination with either of the DQB reverse primers would produce an amplicon size distinguishable from the 67 bp DQA amplicon. The reverse primer A552 has a 25 bp tail to create a 143 bp pair amplification to identify the DQB1*0302 group. Without the 25 bp tail the DQB1*0302 amplicon would only be 128 bp which would be difficult to distinguish reliably from the 105 bp DQB1*02 amplicon.

Fifteen DNA samples from well classified reference cell lines were amplified using the standard protocols for freeze-dried SSP mixes. The primers used in each mix were from the multiplexed Coeliac kit, namely primer IDs A550, A551, A552, A556 and A558. Each mix also contained a pair of primers to amplify a housekeeping gene as a positive control for the PCR (expected size 710 bp). The fifteen amplicons were subject to electrophoresis on the Lab901 screentape DX8 system along with a ladder (bands at 25, 100, 200, 400, 600, 800 and 1,000 bp) which was used as a size reference (FIG. 6).

The following table shows expected results and actual results seen in the testing:

| | | | | | | Results | | |
|---|---|---|---|---|---|---|---|---|
| Lane | DNA Ref | DQA1* | DQA1* | DQB1* | DQB1* | DQ302 | DQ2 | DQA1*05 |
| 2 | 126 | 0105 | 0303 | 050101 | 030302 | | | |
| 3 | 127 | 0103 | 030101 | 030201 | 0603 | + | | |
| 4 | 128 | 010101 | 040101 | 0402 | 050101 | | | |
| 5 | 129 | 010101 | 0505 | 030101 | 050101 | | | + |
| 6 | 130 | 010201 | 0201 | 030302 | 0502 | | | |
| 7 | 131 | 010201 | 0303 | 050201 | 0402 | | | |
| 8 | 132 | 050101 | 0503 | 030101 | | | | + |
| 9 | 133 | 0503 | 0505 | 030101 | | | | + |
| 10 | 134 | 010201 | 0104 | 050201 | 0602 | | | |
| 11 | 135 | 0201 | 0505 | 0202 | 030101 | | + | + |
| 12 | 135 | 0201 | 0505 | 0202 | 030101 | | + | + |
| 13 | 136 | 030101 | 050101 | 0201 | 0302 | + | + | + |
| 14 | 139 | 010201 | 050101 | 0201 | 060401 | | + | + |
| 15 | 140 | 0201 | 0505 | 0202 | 030101 | | + | + |
| 16 | 141 | 010201 | 040101 | 030101 | 050101 | | | |

### Example 6

### RT-PCR

To demonstrate lyophilised RT-PCR in HLA a RT-PCR was designed to specifically identify HLA-B*27. The following standard and Scorpion® primers, specific to a sub-set of HLA-B*27 alleles, were combined with a PCR mastermix, dNTPs, MgCl2 and polymerase, then tested against 100 ng of genomic DNA from a HLA-B*27 positive heterozygous and homozygous sample (DNA samples 1 and 4 respectively) and two HLA-B*27 negative samples; a HLA-B*15:17 sample (DNA sample 2) and a HLA-B*40:01 sample (DNA sample 3) at 2.5 ng/μl.

These were compared to the same PCR reagents in lyophilised format, which were resuspended with the same DNA samples at the same concentration. Positive reactions are expected with the B*27 samples only.

| | | | Binding | |
|---|---|---|---|---|
| ID | Sequence 5′-3′ | Orientation | position | Modification |
| Primer 1 | GGGCTACGTGGACGACATGCT | forward | 167GCT | |
| Primer 2 | CCCATGCCTTGGGACCGGGAGACACAGATG | reverse | 280AAGG | PAM Scorpion |
| | GCATGGG12GTTCTCTCGGTCAGTCTGTGC | | | |
| | CTT | | | |

Each reaction was first soaked at 95 C for 5 minutes, then subject to 45 cycles of 95 C for 10 seconds and 60 C for 15 seconds.

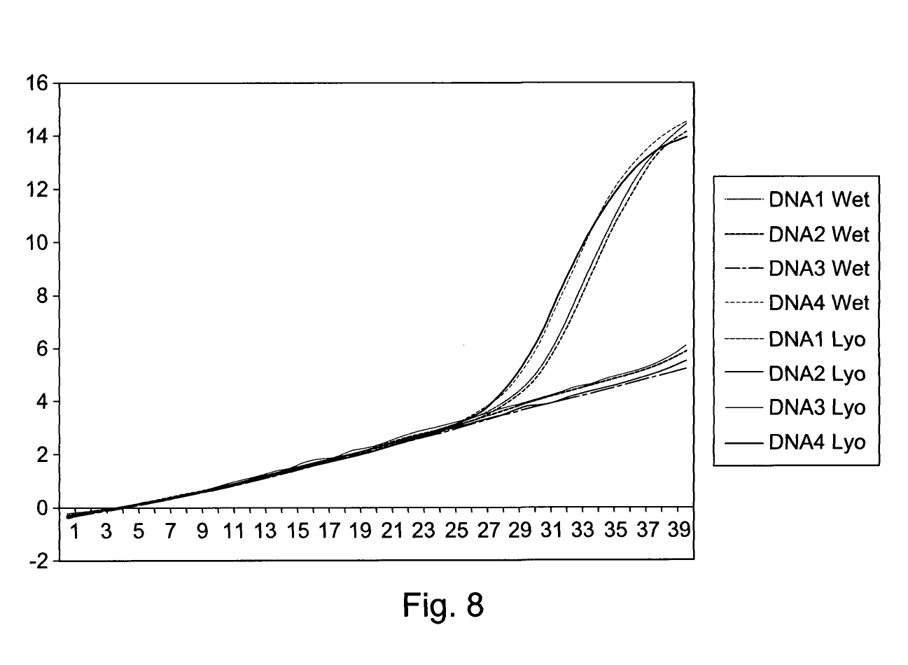

Results were identical in both lyophilised and wet formats—see FIG. 8. The results show the two HLA HLA-B*27-positive samples were amplified, detected and discriminated from non-B*27 alleles in a lyophilised RT-PCR reaction.

### Example 7

### Stability of Lyophilised Compositions

To characterise the stability of the lyophilised mixes at various temperatures a set of eight PCR reactions was created using the previously described PCR buffer mix1 (see Example 1). SSP primers specific for a 198 bp amplification of the HLA-DRB1*0103 allele were set up as shown in table x in 8-reaction PCR strips and stored in individual sealed foil pouches.

TABLE X

| | Control | Allele primer | Allele primer | |
|---|---|---|---|---|
| | amplification | IDs | sequences | Concentration |
| a) | 710 bp DRA | Forward primer | TTGTGGCAGCTTAAGTTTGAAT | 0.3 uM |
| | amplicon | A241 | CCCGCTCGTCTTCCAGGAT | |
| b) | 710 bp DRA | Reverse primer | | 0.2 uM |
| | amplicon | A280 | | |
| c) | 710 bp DRA | | | 0.1 uM |
| | amplicon | | | |
| d) | 710 bp DRA | No allele | | — |
| | amplicon | primers | | |
| e) | 710 bp DRA | Forward primer | TTGTGGCAGCTTAAGTTTGAAT | 0.3 uM |
| | amplicon | A241 | CCCGCTCGTCTTCCAGGAT | |
| f) | 710 bp DRA | Reverse primer | | 0.2 uM |
| | amplicon | A280 | | |
| g) | 710 bp DRA | | | 0.1 uM |
| | amplicon | | | |
| h) | 710 bp DRA | No allele | | — |
| | amplicon | primers | | |

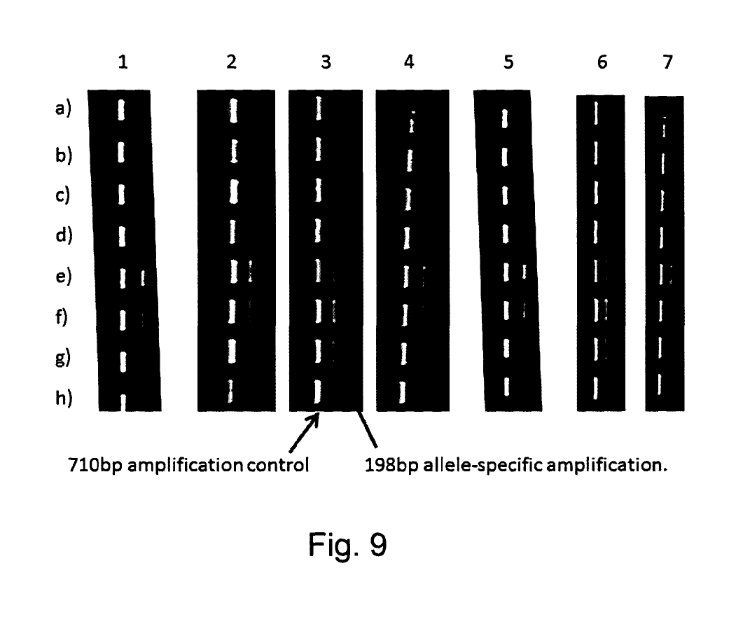

The samples were stored at 2-4° C., ambient (16-25° C.), and at 37° C.: One strip of 8 reactions was tested at day 1, the rest were stored until they were periodically tested by PCR using 100 ng of negative DNA in wells a-d, and 100 ng of a positive DNA in wells e-h). The resulting PCR amplicons were examined by standard horizontal gel electrophoresis as shown in FIG. 9, which is shows the following results:—

### Lane

### 1 Test day 1

2 Test after 12 months at 4° C.
3 Test after 12 months at ambient
4 Test after 12 months at 37° C.
5 Test after 16 months at 4° C.
6 Test after 16 months at ambient
7 Test after 16 months at 37° C.
Tests a-d)=PCR negative sample DNA for HLA-DRB1*0103
Tests e-h)=PCR positive sample DNA for HLA-DRB1*0103

Testing shows the strength of the 700 bp amplicon is undiminished in all reactions at all storage temperatures and times. The positive DNA shows correct and undiminished amplification with the allele-specific primer titration in reactions e-g with no false positives in the negative DNA test in reactions a-e).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A lyophilised composition which is stable at room temperature for use in a reaction for the amplification and/or synthesis of at least one polynucleotide comprising
(i) a polymerase enzyme;
(ii) dNTPs;
(iii) trehalose;
(iv) ammonium sulfate; and
(v) at least one primer.

2. The composition of claim 1 wherein the polymerase enzyme is Taq DNA polymerase.

3. (canceled)

4. The composition of claim 1 wherein the composition is an HLA genotyping composition.

5-7. (canceled)

8. The composition of claim 1 which further comprises a buffering agent selected from the group consisting of Tris-HCl and Tween.

9. (canceled)

10. The composition of claim 1 wherein the at least one primer comprises a template binding region and a tail region wherein the tail region is a nucleotide sequence of between 5 and 100 nucleotides in length.

11-17. (canceled)

18. The composition of claim 1 which comprises at least six primers for amplifying an HLA gene and/or ABO gene and/or an HFE gene.

19. The composition as claimed in claim 18 wherein the HLA gene is HLA-A, HLA-B, HLA-C, HLA-DRB1, DRB3, DRB4, DRB5, DQB1, DQA1, DPA1, DPB1 or any of the polymorphic genes in the MHC cluster on human chromosome 6.

20. The composition of claim 2 which comprises before lyophilisation:
(i) 0.5 units of a Taq DNA polymerase enzyme;
(ii) 0.25 mM dNTPs comprising dATP, dCTP, dTTP. dGTP;
(iii) 5% w/v Trehalose di hydrate as a cryprotectant;
(iv) 0.2M ammonium sulphate;
(v) at least one genotyping primer; and
(vi) the buffering agents Tris-HCl and Tween,
wherein the at least one primer comprises a template binding region and a tail region.

21. The composition of claim 1 further comprising Carbowax 20M.

22. The composition of claim 1 further comprising a reporter reagent.

23. The composition of claim 1 that is room temperature stable for at least 12 months.

24. A method for detecting the presence or absence of at least one variant nucleotide in one or more nucleic acids contained in an aqueous sample, comprising, preparing solution containing
(i) a polymerase enzyme;
(ii) dNTPs;
(iii) trehalose di-hydrate;
(iv) ammonium sulfate; and
(v) at least one primer,
lyophilising the solution,
storing the solution at room temperature,
adding the one or more nucleic acids in an aqueous sample to the lyophilised solution, and amplifying the one or more nucleic acids to produce extension products and detecting the presence or absence of the suspected variant nucleotide from the presence or absence of an extension product.

25. The method of claim 24 wherein the primer is, a diagnostic primer for a diagnostic portion of a target base sequence of the nucleic acids under hybridising conditions, the nucleotide sequence of the diagnostic primer being such that it is substantially complementary to the diagnostic portion, the 5′ or 3′ terminal nucleotide of the diagnostic primer being either complementary to the suspected variant nucleotide or to the corresponding normal nucleotide, whereby an extension product of the diagnostic primer is synthesised when the said terminal nucleotide of the diagnostic primer is complementary to the corresponding nucleotide in the target base sequence, no extension product being synthesised when the terminal nucleotide of the diagnostic primer is not complementary to the corresponding nucleotide in the target base sequence.

26. (canceled)

27. An HLA and/or ABO and/or HFE genotyping kit comprising a lyophilised composition for the amplification and/or synthesis of at least one polynucleotide comprising:
(i) a Taq DNA polymerase enzyme;
(ii) at least one nucleotide selected from the group consisting of dATP, dCTP, dTTP, dGTP and dUTP;
(iii) trehalose dihydrate;
(iv) ammonium sulphate;
(v) at least one genotyping primer; and
(vi) optionally a buffering agent,
wherein the at least one primer comprises a template binding region and a tail region.

28. The kit of claim 27 wherein the at least one nucleotide comprises a thermally labile 3′-substitution group.

29-32. (canceled)
